(19) United States
(12) Patent Application Publication
(10) Pub. No.: US 2018/0319527 A1
(43) Pub. Date: Nov. 8, 2018

(54) DEVICE AND METHOD FOR STERILIZATION OF A SHEET OF PACKAGING MATERIAL, AND FILLING MACHINE

(71) Applicant: Tetra Laval Holdings & Finance S.A., Pully (CH)

(72) Inventor: Roberto Ghirardello, Carpi (IT)

(21) Appl. No.: 15/774,017

(22) PCT Filed: Nov. 16, 2016
(86) PCT No.: PCT/EP2016/077856

§ 371 Date: May 7, 2018

(30) Foreign Application Priority Data
Nov. 23, 2015 (EP) 15195789.1

(51) Int. Cl. B65B 55/02; B65B 55/08; B65B 55/10; A61L 2/08; A61L 2/20; A61L 2/26

(52) U.S. Cl. CPC B65B 55/027; B65B 55/08; B65B 55/103; A61L 2/087; A61L 2202/15; A61L 2/26; A61L 2202/122; A61L 2202/121; A61L 2202/23; A61L 2/208

(57) ABSTRACT

The invention relates to a device (1) for sterilization of a sheet of packaging material (2). The device (1) comprises a first chamber (3) having an inlet (4) and an outlet (5) for the sheet of packaging material (2), and a second chamber (6) having an inlet (5) and an outlet for the sheet of packaging material (2). The outlet (5) of the first chamber is connected to the inlet (5) of the second chamber (6). The first chamber (3) comprises an element (8) which is movable between a first position, in which the device (1) is in a mode for sterilization of the sheet of packaging material (2), and a second position, in which the inlet (4) of the first chamber (3) is substantially closed and the device (1) is in a mode for sterilization of the second chamber (6). The invention also relates to a filling machine for filling a packaging material (2) with a food product, and a method for sterilizing a sheet of packaging material.

### TECHNICAL FIELD

The invention relates to a device and a method for sterilization of a sheet of packaging material, and a filling machine.

### BACKGROUND ART

In the prior art several different devices and methods for sterilizing packaging material are known, in particular for packaging material intended to form containers. One method widely used in the prior art is sterilization by means of a hydrogen peroxide bath. However, efforts are being made to reduce the use of chemicals when sterilizing the packaging material. Therefore, devices and methods have also become known which sterilize packaging material by means of radiation, in particular by means of electron beams. In general, there is provided an emitter that is adapted to emit charge carriers, in particular electrons, wherein the packaging material can be sterilized by the charge carriers. For this purpose the packaging material and the emitter or the emitters, respectively, are moved relatively to each other.

This type of sterilization by means of radiation is conventionally conducted in a radiation chamber of a sterilization device. The radiation chamber itself needs to be sterilized before it can be used for sterilization of the packaging material, normally upon production start every morning. In order to sterilize the radiation chamber, a flow of vapour of hydrogen peroxide is introduced into the radiation chamber.

The need is felt to limit or completely avoid emission of the vapour of hydrogen peroxide into the surroundings of the sterilization devices.

### SUMMARY OF THE INVENTION

It is an object of the present invention to mitigate, alleviate or eliminate one or more of the above-identified deficiencies in the art and disadvantages singly or in any combination and solve at least the above mentioned problem.

According to a first aspect of the invention, these and other objects are achieved in full, or at least in part, by a device for sterilization of a sheet of packaging material, particularly a device for sterilization of a sheet of packaging material by means of electron beam. The device comprises a first chamber having an inlet and an outlet for the sheet of packaging material, and a second chamber having an inlet and an outlet for the sheet of packaging material. The outlet of the first chamber is connected to the inlet of the second chamber. The first chamber comprises an element which is movable between a first position, in which the device is in a mode for sterilization of the sheet of packaging material, and a second position, in which the inlet of the first chamber of the device is substantially closed and the device is in a mode for sterilization of the second chamber of the device. In this way, the inlet of the device, i.e. the inlet of the first chamber of the device, is prevented from leaking vapour of hydrogen peroxide out of the device and into the surrounding environment during sterilization of the second chamber of the device. This is very important since there are normally regulations in this regards which, among other things, include a maximum allowed value of the amount of the vapour of hydrogen peroxide in the surrounding environment of the device.

In a preferred embodiment of the present invention, the movable element may be constituted by a flap which is moved by means of an actuator device, particularly a pneumatic cylinder. This is advantageous in that it is a simple but reliable solution on how to open and close the inlet of the first chamber.

The second chamber may further comprise a vapour inlet through which the vapour of hydrogen peroxide is introduced when the second chamber is to be sterilized, i.e. when the movable element is in its second position.

The first chamber may further comprise a vapour outlet through which vapour is sucked out of the device by means of a suction pipe which is connected to the vapour outlet.

When the device is operated for sterilizing the sheet of packaging material, the movable element is arranged in its first position so that the inlet of the suction chamber is open.

When the second chamber of the device is to be sterilized, the movable element is arranged in its second position so that the inlet of the first chamber is closed. In this configuration, the second chamber is sterilized and the vapour of hydrogen peroxide is sucked away through the vapour outlet and the suction pipe.

The first chamber may be a suction chamber and the second chamber may be an electron beam irradiation chamber, particularly a low voltage electron beam irradiation chamber. It is in the radiation chamber that the sterilization of the sheet of packaging material takes place by using a low voltage electron beam. Vapour of hydrogen peroxide is preferably used for sterilization of the radiation chamber of the device. This process is conducted when the device is in its mode for sterilization of the radiation chamber, and thus the movable element is in its second position to prevent vapour of hydrogen peroxide from escaping out of the inlet of the suction chamber.

Generally, all terms used in the claims are to be interpreted according to their ordinary meaning in the technical field, unless explicitly defined otherwise herein. All references to “a/an/the [element, device, component, means, step, etc.]” are to be interpreted openly as referring to at least one instance of the element, device, component, means, step, etc., unless explicitly stated otherwise.

### BRIEF DESCRIPTION OF THE DRAWINGS

The above objects, as well as additional objects, features and advantages of the present invention, will be more fully appreciated by reference to the following illustrative and non-limiting detailed description of preferred embodiments of the present invention, when taken in conjunction with the accompanying drawings, wherein:

FIG. 1 is a cross sectional view of an exemplary embodiment of a device according to the invention when in a mode for sterilization of a sheet of packaging material.

FIG. 2 is a cross sectional view of the device when in a mode for sterilization of the second chamber of the device.

FIG. 3 is a cross sectional view of the second chamber of the device.

FIG. 4 is an enlarged view of a portion of the device in FIG. 1 and FIG. 2 and of a movable element arranged within the device.

FIG. 5 is a block diagram of an exemplary embodiment of a method for sterilization of a sheet of packaging material according to the invention.

### DETAILED DESCRIPTION OF PREFERRED EMBODIMENTS OF THE INVENTION

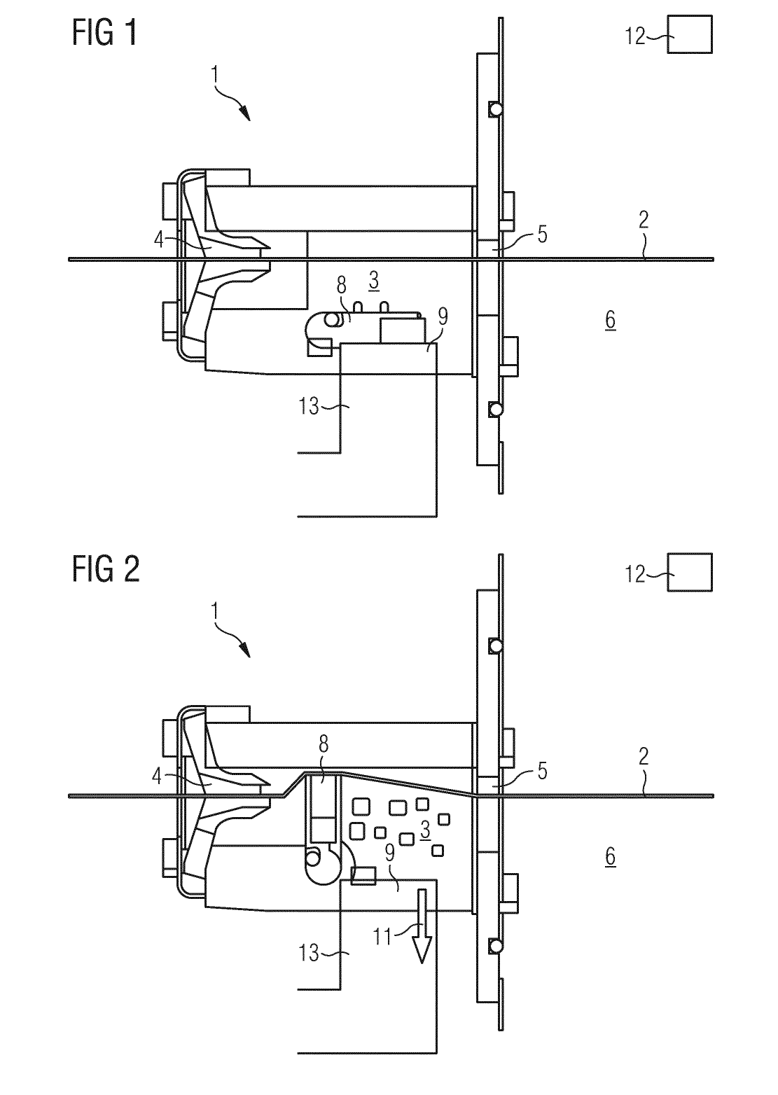

FIG. 1 and FIG. 2 illustrate a device 1 for sterilization of a sheet of packaging material 2 with electron beam according to an exemplary embodiment of the invention. The device 1 has a first chamber 3 which is constituted by a suction chamber and a second chamber 6 which is constituted by a radiation chamber, i.e. a sterilization chamber. The suction chamber 3 has an inlet 4 and an outlet 5, and the radiation chamber 6 has an inlet 5 and an outlet. The outlet 5 of the suction chamber 3 is connected to the inlet 5 of the radiation chamber 6. Thus, the sheet of packaging material 2 can be introduced from the exterior of the device 1 into the suction chamber 3 via its inlet 4, travel through the suction chamber 3 and out of the outlet 5 into the radiation chamber 6 via its inlet 5. The suction chamber 3 further comprises a vapour outlet 9 which is connected to a suction pipe 13, and a movable element 8. The movable element 8 is constituted by a flap which is movable between a first position (FIG. 1), in which the device 1 is in a mode for sterilization of the sheet of packaging material 2 (conducted in the radiation chamber 6), and a second position (FIG. 2), in which the inlet 4 of the suction chamber 3 is substantially closed and the device 1 is in a mode for sterilization of the radiation chamber 6.

When the device 1 is in the mode for sterilization of the sheet of packaging material 2, the sheet of packaging material 2 travels through the device 1 and is sterilized by means of a low voltage electron beam (LVEB) device 15. Here, the flap 8 is arranged in its first position (FIG. 1). However, when the radiation chamber 6 needs to be sterilized (normally upon start of the device 1 in the morning), the flap 8 is placed in its second position and the inlet 4 of the suction chamber 3 is thereby substantially closed. Thereafter, vapour of hydrogen peroxide is introduced directly into the radiation chamber 6 through a vapour inlet 12 to sterilize the chamber 6. Simultaneously, the vapour outlet 9 in the suction chamber 3 provides suction via the suction pipe 13 which is connected to a vacuum. This is done in order to suck the residues of the vapour of hydrogen peroxide out of the radiation chamber 6. Further, since the flap 8 seals off the inlet 4 of the suction chamber 3, the residues of the vapour of hydrogen peroxide cannot escape out that way and into the surroundings of the device 1.

As stated above, the vapour used for sterilizing the radiation chamber 6 of the device 1 is preferably hydrogen peroxide. Hydrogen peroxide is a very effective gas for sterilization, but has to be handled carefully. The flap 8 has the purpose of sealing the inlet 4 of the suction chamber 3, when it is in its second position (FIG. 2), so that the gas is prevented from exiting the device 1 during the sterilization of the radiation chamber 6, or at least so that only an acceptable amount of gas is allowed to exit the device 1 during sterilization of the radiation chamber 6.

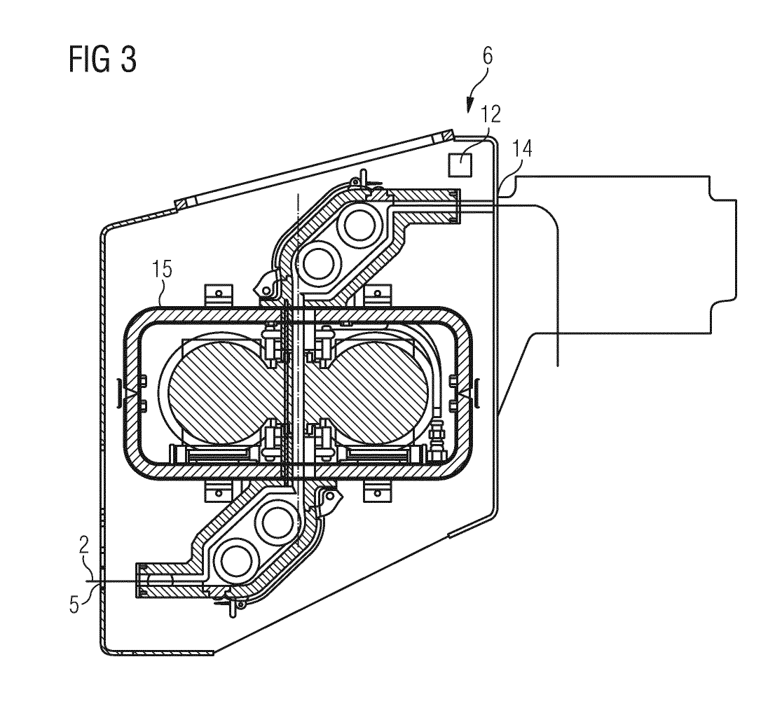

FIG. 3 illustrates the radiation chamber 6 of the device 1. As stated above, the radiation chamber 6 has an inlet 5 and an outlet 14 for the sheet of packaging material 2. The sheet of packaging material 2 enters into the radiation chamber 6 through the inlet 5, passes the low voltage electron beam (LVEB) device 15 where it is sterilized on both sides by electron beam emitters, and exits the radiation chamber 6 through the outlet 14. There is a constant overpressure present in the radiation chamber 6 so that dirty, i.e. unsterile, air is prevented from entering into the radiation chamber 6 during the sterilization of the sheet of packaging material 2 and also during sterilization of the radiation chamber 6.

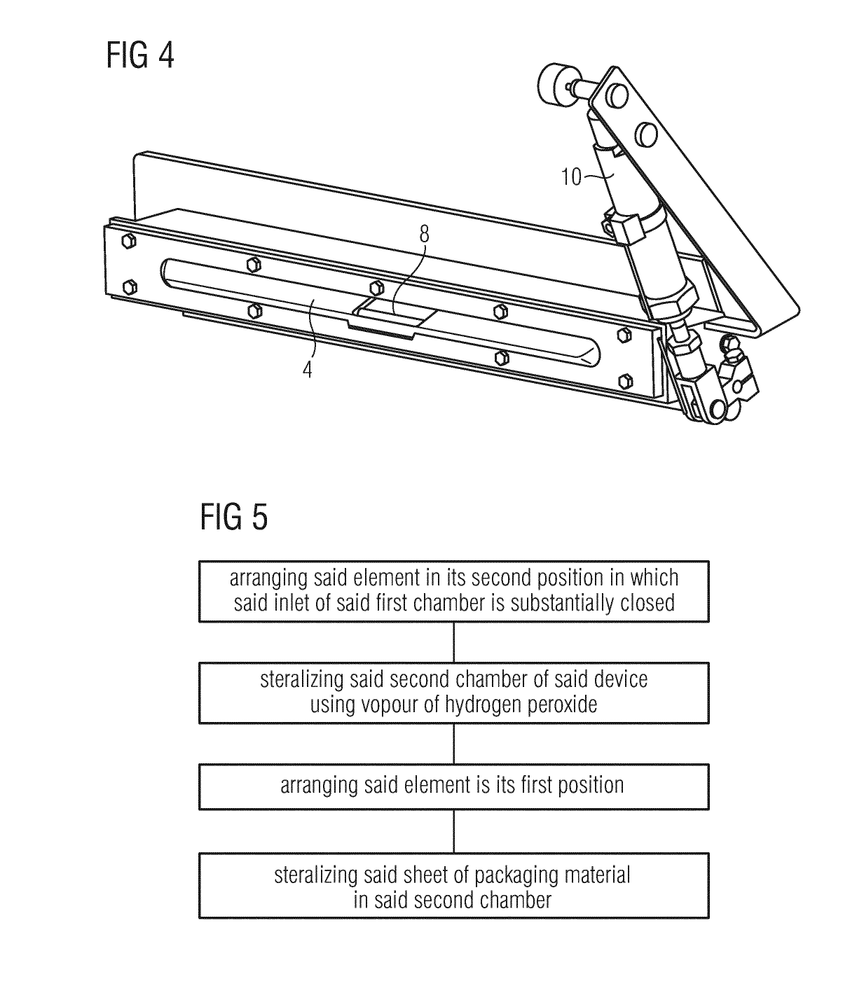

FIG. 4 is a perspective view of a portion of the device 1. Here, the flap 8 and the inlet 4 of the second chamber 3 are specifically illustrated. In FIG. 4, it can be seen that the flap 8 is operated and moved by means of a pneumatic cylinder 10.

FIG. 5 is a block diagram illustrating the steps of the method of sterilizing first the radiation chamber and then the packaging material in the device of FIGS. 1 to 4.

Firstly, upon start of the device, the flap is arranged in its second position. Here, the inlet of the suction chamber is substantially closed. Now, the radiation chamber can be sterilized by means of vapour of hydrogen peroxide entering through the vapour inlet. Thereafter, the flap is arranged in its first position so that the device is in the mode for sterilization of the sheet of packaging material. And finally, the sheet of packaging material is sterilized in the radiation chamber.

It is understood that other variations in the present invention are contemplated and in some instances, some features of the invention can be employed without a corresponding use of other features. Accordingly, it is appropriate that the appended claims be construed broadly in a manner consistent with the scope of the invention.

1-10. (canceled)

11. A device for sterilization of a sheet of packaging material, the device comprising:
a first chamber having an inlet and an outlet for the sheet of packaging material, and
a second chamber having an inlet and an outlet for the sheet of packaging material,
wherein the outlet of the first chamber is connected to the inlet of the second chamber,
wherein the first chamber further includes a moveable element that is movable between a first position, associated with a mode for sterilizing the sheet of packaging material, and a second position, in which the inlet of the first chamber is substantially closed and associated with a mode for sterilizing the second chamber.

12. The device according to claim 11, wherein the movable element includes a flap.

13. The device according to claim 12, further including a pneumatic cylinder configured to move the flap.

14. The device according to claim 11, wherein the first chamber further comprises a vapor outlet through which vapor is suctioned.

15. The device according to claim 14, wherein a suction pipe is connected to the vapor outlet.

16. The device according to claim 11, wherein the second chamber further comprises a vapor inlet.

17. The device according to claim 11, wherein the first chamber is a suction chamber and the second chamber is an electron beam irradiation chamber.

18. The device according to claim 11, wherein the mode for sterilizing the second chamber includes using vapor of hydrogen peroxide to sterilize the second chamber and preventing the vapor of hydrogen peroxide from escaping through the inlet of the first chamber.

19. A filling machine for filling a packaging material with a food product, comprising a device according to claim 11 for sterilizing the sheet of packaging material.

20. A method for sterilizing a sheet of packaging material by use of a sterilization device, wherein the device includes a first chamber having an inlet and an outlet for the packaging material and a moveable element movable between a first position and a second position, and a second chamber having an inlet and an outlet for the packaging material, wherein the outlet of the first chamber is connected to the inlet of the second chamber, wherein the method comprises:
moving the moveable element to the second position to substantially close the inlet of the first chamber;
sterilizing the second chamber with a vapor of hydrogen peroxide;
moving the moveable element to the first position; and
sterilizing the sheet of packaging material in the second chamber.
